(19) United States
(12) Plant Patent Application Publication
(10) Pub. No.: US 2002/0148017 P1
(43) Pub. Date: Oct. 10, 2002

(54) Blackberry plant named 'Sleeping Beauty'

(76) Inventor: Carlos D. Fear, Aptos, CA (US)

Correspondence Address: PENNIE AND EDMONDS, 1155 AVENUE OF THE AMERICAS, NEW YORK, NY, 100362711

(21) Appl. No.: 09/772,329

(22) Filed: Jan. 29, 2001

(51) Int. Cl. A01H 5/00

(52) U.S. Cl. PLT/203

(57) ABSTRACT

The present invention relates to a new and distinct cultivar of blackberry plant named ‘Sleeping Beauty’. The new cultivar is distinguished from other blackberry cultivars by its large fruit, low chill requirement, and long fruiting period. The new cultivar is distinguished from its pollen parent by having an earlier fruiting period and much lower chill requirement. The new cultivar is distinguished from its seed parent by having firmer and less acidic fruit.

### 1. BACKGROUND OF THE INVENTION

This invention relates to a new cultivar of blackberry called ‘Sleeping Beauty’. The new cultivar was developed from hybridization of the unpatented female cultivar ‘Brazos’ with the unpatented male cultivar ‘Hull Thornless’. The parents were crossed in Spring 1991 whereafter fruit and seed were collected to produce seedlings for field planting in Watsonville, Calif. in 1992. The new cultivar was selected in 1994 for its early fruiting period and large fruit size. The cultivar has been asexually propagated, and reproduced true to type plants by in vitro shoot tip culture.

### 2. SUMMARY OF THE INVENTION

The present invention provides a new and distinct blackberry cultivar named ‘Sleeping Beauty’. The variety is botanically identified as Rubus L. subgenus Rubus. The new cultivar produces a floricane crop which begins in mid-May and continues until mid-August. The new blackberry variety is distinguished from other varieties by a number of characteristics as set forth in Table 1. In particular, the new cultivar is distinguished by its large fruit, low chill requirement and long fruiting period. Yield of the new cultivar is moderate to high when compared to many other varieties.

### 3. COMPARISON TO SIMILAR VARIETIES

The variety that we believe to be similar to ‘Sleeping Beauty’ from those known to us is ‘Brazos’, an unpatented variety. ‘Sleeping Beauty’ is particularly different from ‘Brazos’ by having firmer, less acidic fruit and by being slightly smaller in size.

### 4. BRIEF DESCRIPTION OF THE DRAWINGS

The accompanying photographs show typical specimens of the fruit, leaves and shoot of the new cultivar, in color as nearly true as reasonably possible in color illustrations of this type.

FIG. 1 is a photograph showing a primocane shoot, mature leaf and stem of ‘Sleeping Beauty’.

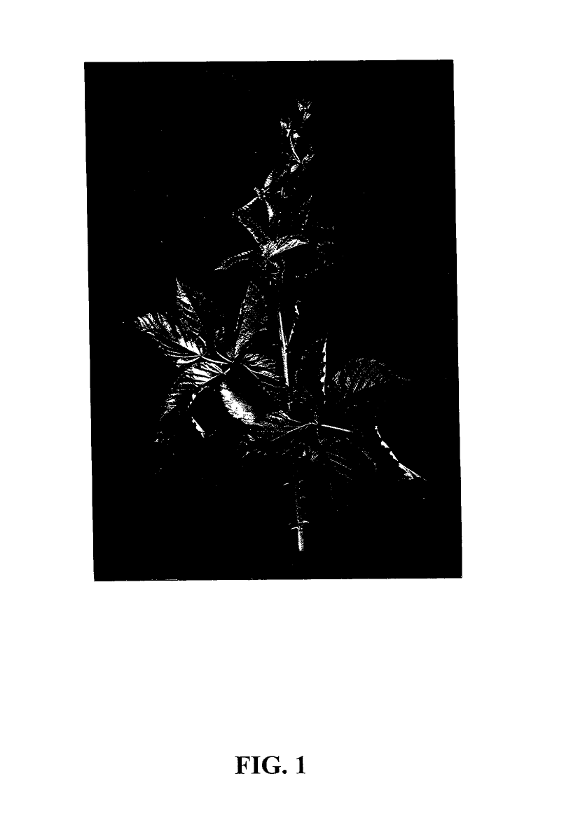

FIG. 2 is a photograph showing a close-up view of a primocane shoot, mature leaf and stem of ‘Sleeping Beauty’.

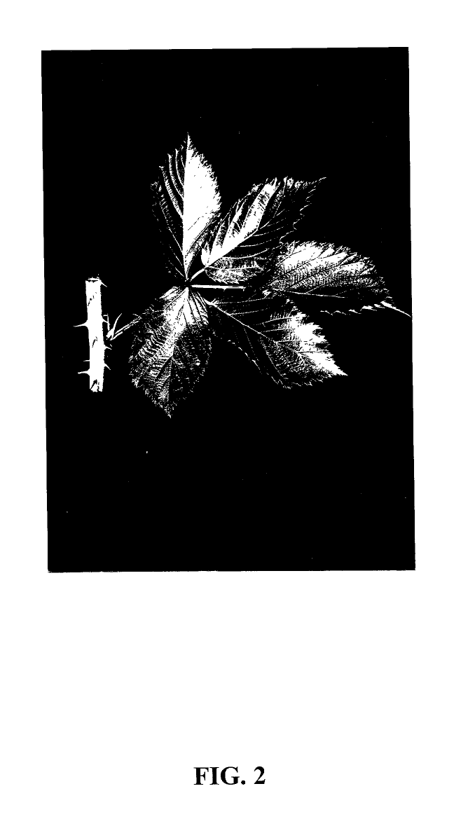

FIG. 3 is a photograph showing the flowering and fruiting laterals of ‘Sleeping Beauty’.

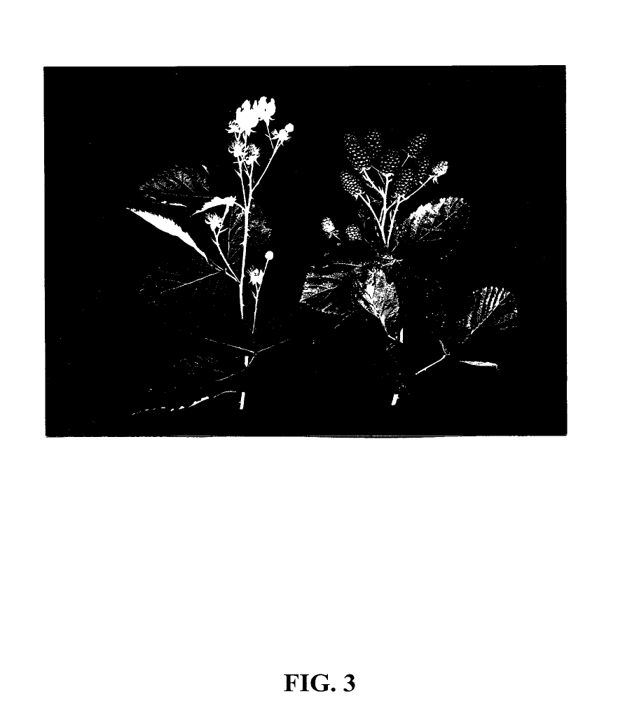

### 5. DESCRIPTION OF THE NEW VARIETY

The following detailed description of the new blackberry cultivar, ‘Sleeping Beauty’, is based upon recorded observations of plants and fruit grown between 1996 and 2000 in Watsonville, Calif., and is believed to apply to plants of the ‘Sleeping Beauty’ cultivar grown in similar conditions of soil and climate elsewhere. This description is in accordance with terminology used by the International Union for the Protection of New Varieties of Plants (UPOV). Throughout this specification, color names beginning with a small letter signify that the name of the color, as used in common speech, is aptly descriptive. Color data beginning with a capital letter and followed by an alphanumeric code indicate the most similar color designations as provided by The Royal Horticultural Society (RHS) Colour Chart published by The Royal Horticultural Society of London, England. Color designations, color descriptions, and other phenotypical descriptions may deviate from the stated values and descriptions depending upon variation in environmental, seasonal, climatic and cultural conditions.

### 5.1 CHARACTERISTICS OF THE NEW VARIETY

Table 1 provides information on the plant and fruit characteristics of the new blackberry cultivar ‘Sleeping Beauty’ compared with characteristics of the unpatented blackberry cultivars ‘Olallie’ and ‘Chester’. Both ‘Olallie’ and ‘Chester’ are currently important cultivars for fresh market shipping, and thus are comparable to the proposed use of the new invention, ‘Sleeping Beauty’. Observations of ‘Sleeping Beauty’ and ‘Olallie’ and ‘Chester’ were taken in side-by-side comparison in 1999 and 2000.

The new blackberry cultivar is particularly characterized and distinguished from other cultivars by its low chill requirement and its long fruiting cycle. Canes of ‘Sleeping Beauty’ are vigorous and thorny.

The fruit color of ‘Sleeping Beauty’ is black with moderate amount of post harvest drupelet color reversion. The fruit is large with a slightly tart flavor. Postharvest fruit rot resistance is average in comparisons with many selections and varieties.

‘Sleeping Beauty’ is distinguished from its pollen parent, ‘Hull Thornless’, by being thorny, having a more upright growth habit, and having larger fruit. Additionally, ‘Sleeping Beauty’ has a much lower chill requirement and an earlier season than its pollen parent ‘Hull Thornless’. ‘Sleeping Beauty’ is distinguished from its seed parent, ‘Brazos’, by having firmer and less acidic fruit.

TABLE 1
PLANT CHARACTERISTICS OF ‘SLEEPING BEAUTY’

| | Sleeping | | |
|---|---|---|---|
| | Beauty | Olallie | Chester |
| GENERAL | | | |
| Vigor | high | Moderate - | high |
| | | high | |
| Growth habit | upright | trailing | semi-upright |
| Productivity | moderately | high | high |
| | high | | |
| Self fruitfulness | yes | yes | yes |
| Number of young | many | medium | medium |
| shoots | | | |
| CANES | | | |
| Primocanes | | | |
| Anthocyanin | absent | present | present |
| coloration | | | |
| Spines | present | present | absent |
| color | green | purple | — |
| attitude of tip | horizontal to | horizontal | — |
| | downward | | |
| texture | rigid | heavy | — |
| presence and | present; | present; | absent |
| distribution | irregularly | irregularly | |
| on petioles | distributed | distributed | |
| density in central third | medium | medium | — |
| of shoot | | | |
| Internodal distance | 3.3 | 2.6 | 3.1 |
| (cm) - central third | | | |
| of mature cane | | | |
| Glaucosity on full | absent or very | weak | weak |
| grown shoot | weak | | |
| Strength of full | strong | medium | strong |
| grown shoot | | | |
| Cane cross section | angular to | rounded to | angular to |
| | grooved | angular | grooved |
| LEAVES | | | |
| Relief between veins | weak | medium | medium |
| Number of leaflets | usually 5 | usually 3 | usually 5 |
| Leaf color | medium | medium | light |
| upper side | 139A | 137A, 137B | 147A |
| underside | 137C | 147B | 146A |
| Glossiness of upper | glossy | medium | dull |
| surface | | | |
| Leaf cross section | concave | concave-flat | concave |
| Terminal leaflet | | | |
| length (cm) | 9.5 | 8.9 | 11.1 |
| width (cm) | 6.5 | 7.6 | 9 |
| shape | ovate | cordate | cordate |
| tip | acuminate | acuminate | acuminate |
| base | rounded | cordate | cordate |
| margin | double serrate | double serrate | double serrate |
| Lateral leaflet | | | |
| overlap of lateral | touching | overlapping | overlapping |
| leaflets | | | |
| length (cm) | 9.1 | 8.7 | 10.2 |
| width (cm) | 5.4 | 6.1 | 7.1 |
| shape | ovate | ovate | ovate |
| tip | acute | acuminate | acute |
| base | acute | acute | acute |
| margin | double serrate | double serrate | serrate |
| Petiole | | | |
| mean length (cm) | 6.8 | 5.3 | 7.9 |
| range | 5.6-7.8 | 3.6-8.7 | 3.9-10.2 |
| pigmentation of upper | purplish red | green - slightly | purple |
| surface | | pink | |
| pigmentation of | some red, | green - slightly | green - pinkish |
| underside | mostly green | pink | |
| Length of stalklet | short | very short | medium |
| Rachis length (cm) | 2.9 | 2.8 | 3.1 |
| between terminal and | | | |
| adjacent lateral | | | |
| leaflets) | | | |
| Stipule orientation | erect | variable; | erect |
| | | clasping to | |
| | | erect | |
| FLOWERS | | | |
| Time of bud burst | early | early | late |
| Time of beginning | early | early | late |
| of flowering | | | |
| Flower size | large | small to | small to |
| | | medium | medium |
| Petal size | | | |
| length (mm) | 20.1 | 16.5 | 18.3 |
| width (mm) | 16.5 | 11.7 | 10.9 |
| Anthocyanin color | absent | absent | present |
| of pedicel | | | |
| Intensity of pedicel | — | — | weak |
| coloration | | | |
| Length of pedicel | medium-long | long | short |
| Flower number (third | 7.6 | 3.6 | 2 |
| node from tip of | | | |
| lateral) | | | |
| FRUIT | | | |
| Harvest season | early to mid | early | mid to late |
| Dimensions | 5.1 | 5.2 | 3.2 |
| weight (g/fruit) | | | |
| size | medium | medium | small |
| length (cm) | 2.4 | 3.3 | 1.9 |
| width (cm) | 1.7 | 1.4 | 1.9 |
| Fruiting lateral length | medium-long | medium | medium - long |
| (in mid cane) | | | |
| mean number of fruit | 9.1 | 6.2 | 22.8 |
| per lateral | | | |
| range | 4-18 | 3-9 | 17-40 |
| Shape | elliptic | narrow ovate | round to ovate |
| | longer than | much longer | as long as |
| | broad | than broad | broad |
| Color | black | purple-black to | black |
| | | black | |
| immature | 187B | 178A-183B | 184A |
| maturing | 200A | 187A | 200A-202A |
| mature | 202A | 200A | 202A |
| Firmness | medium | medium | firm |
| Glossiness | medium strong | medium - | medium |
| | | strong | |
| Soluble solids | 10.2 | 9.7 | 9.9 |
| Titratable acidity | 10.2 | 13.3 | 9.9 |
| (% as citric acid) | | | |
| (ml of added 0.1 N | | | |
| NaOH to pH 8.1) | | | |
| Number of drupelets | 94 | 86 | 40 |
| per fruit | | | |

### 5.2 NUCLEIC ACID FINGERPRINTING

Distinctive patterns of polymorphism can be detected using a variety of nucleic acid analysis methods. In one non-limiting example, molecular genetic maps can be produced using random amplified polymorphic DNA (RAPD) (Williams et al., 1990, “DNA polymorphisms amplified by arbitrary primers are useful as genetic markers”, Nucleic Acids Res. 18(22):6531-5). Using a variety of oligonucleotide primers, alone or in combination, RAPD analysis of Sleeping Beauty, Chester, and Olallie yielded DNA fragment patterns that uniquely distinguish each of these genetically distinct genotypes.

We claim:

1. A new and distinctive cultivar of blackberry plant, substantially as shown and described.
